(19) United States
(12) Patent Application Publication
(10) Pub. No.: US 2021/0402152 A1
(43) Pub. Date: Dec. 30, 2021

(54) COILED VASCULAR ACCESS INSTRUMENT AND RELATED SYSTEMS AND METHODS

(71) Applicant: Becton, Dickinson and Company, Franklin Lakes, NJ (US)

(72) Inventors: Jonathan Karl Burkholz, Salt Lake City, UT (US); Weston F. Harding, Lehi, UT (US); Curtis H. Blanchard, Riverton, UT (US); Megan Scherich, Salt Lake City, UT (US)

(21) Appl. No.: 17/339,505

(22) Filed: Jun. 4, 2021

Related U.S. Application Data
(60) Provisional application No. 63/045,559, filed on Jun. 29, 2020.

(51) Int. Cl. A61M 25/06; A61M 25/09

(52) U.S. Cl. CPC A61M 25/0606; A61M 2025/09083; A61M 25/09

(57) ABSTRACT

A vascular access instrument may be configured to insert through a vascular access device, such as, for example, a catheter assembly. The vascular access instrument may include a wire, which may be monolithically formed as a single unit. The wire may include a coil portion, which may include multiple loops wound around a central axis. The wire may include a core portion extending through the coil portion and aligned with the central axis. In some embodiments, the wire may include a bent portion connecting a distal end of the coil portion with a distal end of the core portion. The bent portion may be blunt and atraumatic.

### RELATED APPLICATIONS

This application claims benefit of U.S. Provisional Patent Application No. 63/045,559, filed on Jun. 29, 2020, entitled COILED VASCULAR ACCESS INSTRUMENT AND RELATED SYSTEMS AND METHODS, which is incorporated herein in its entirety.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle peripheral intravenous (“IV”) catheter. In this case, the catheter may be mounted over an introducer needle having a sharp distal end. The catheter and the introducer needle may be assembled so that the distal end of the introducer needle extends beyond the distal end of the catheter with the bevel of the needle facing up away from skin of the patient. The catheter and the introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using the catheter may be difficult for several reasons, particularly when a dwell time of the catheter within the vasculature is more than one day. When the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, the catheter is often used for acquiring a blood sample at a time of catheter placement, but the catheter is less frequently used for acquiring a blood sample during the catheter dwell period. Therefore, when a blood sample is required, an additional needle stick is often used to provide vein access for blood collection, which may be painful for the patient and result in higher material costs.

In some instances, in order to avoid the additional needle stick, a vascular access instrument may be used to access the vasculature of the patient via the catheter. The vascular access instrument may be inserted through the catheter and into the vasculature to extend a life of the catheter and allow blood withdrawal through the catheter without the additional needle stick.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices. More particularly, the present disclosure relates to a vascular access instrument, which may be delivered through a catheter assembly to vasculature of a patient via an instrument advancement device. In some embodiments, the vascular access instrument may facilitate an increased dwell period of a catheter of the catheter assembly within the vasculature of the patient. In some embodiments, the instrument advancement device may be used to advance the vascular access instrument into the catheter and/or beyond a distal end of the catheter when the catheter is compromised to overcome obstructions such as thrombus, valves, and/or a fibrin sheath in or around the catheter that may otherwise prevent blood draw. In some embodiments, the instrument advancement device may provide needle-free delivery of the vascular access instrument to the vasculature of the patient for blood collection, fluid delivery, patient or device monitoring, or other clinical needs by utilizing an existing catheter dwelling within the vasculature.

In some embodiments, the vascular access instrument may be configured to insert through a vascular access device, such as, for example, the catheter assembly. In some embodiments, the vascular access instrument may include a wire, which may be monolithically formed as a single unit. In some embodiments, the wire may include a coil portion, which may include multiple loops wound around a central axis. In some embodiments, the wire may include a core portion extending through the coil portion and aligned with the central axis. In some embodiments, the wire may include a bent portion connecting a distal end of the coil portion with a distal end of the core portion.

In some embodiments, the bent portion may form a distal end of the wire. In some embodiments, the bent portion may be disposed distal to the coil portion. In some embodiments, the distal end of the wire may be blunt. In some embodiments, the bent portion may include a U-shape. In some embodiments, the bent portion may include a loop.

In some embodiments, the coil portion may be formed by a flat portion of the wire wound around the central axis into the loops. As referred to in the present disclosure, the term “flat portion of the wire” may correspond to a portion of the wire that includes a first side and a second side opposite the first side, and the first side and/or the second side is planar prior to the wire being wound around the central axis into loops during manufacture. In some embodiments, the first side may form an outer surface of the coil. In some embodiments, the second side may form an inner surface of the coil portion. In some embodiments, the core portion may contact the inner surface of the coil portion.

In some embodiments, each of the loops of the coil portion may be spaced apart from a next adjacent loop of the loops. In some embodiments, each of the loops of the coil portion may contact the next adjacent loop of the loops around a circumference of the next adjacent loop. In some embodiments, the coil portion and/or the core portion may be cylindrical. In some embodiments, the core portion may be flat. In some embodiments, the core portion may be offset from the central axis. In some embodiments, a proximal end of the coil portion may be joined to the core portion. In some embodiments, the coil portion may be tapered. In some embodiments, a proximal end of the core portion may taper outwardly in a proximal direction.

In some embodiments, the wire may include a straight portion connected to a proximal end of the coil portion. In some embodiments, the straight portion may be parallel to a proximal end of the core portion. In some embodiments, an inner portion of the straight portion and an inner portion of the proximal end of the core portion may be joined together. In some embodiments, the inner portion of the straight portion and the inner portion of the proximal end of the core portion may not be joined together.

In some embodiments, the vascular access instrument may include another wire coupled to the straight portion and the proximal end of the core portion. In some embodiments, the other wire may include nitinol or stainless-steel. In some embodiments, a distal end of the other wire may be disposed proximal to the coil portion.

In some embodiments, the vascular access instrument may include a tube surrounding the straight portion and/or the proximal end of the core portion. In some embodiments, the tube may include nitinol or stainless-steel. In some embodiments, a distal end of the tube may be disposed proximal to the coil portion. In some embodiments, the tube may be joined to the straight portion and/or the proximal end of the core portion.

In some embodiments, a vascular access system may include the catheter assembly, which may include a catheter adapter and a catheter extending distally from the catheter adapter.

In some embodiments, an instrument advancement device may be coupled to the catheter assembly. In some embodiments, the instrument advancement device may include the vascular access instrument. In some embodiments, the instrument advancement device may be configured to advance the vascular access instrument from a retracted position to an advanced position beyond a distal end of the catheter. In some embodiments, the distal end of the catheter may include a distal opening. In some embodiments, the coil portion may extend through the distal opening of the catheter in response to the vascular access instrument being in the advanced position.

It is to be understood that both the foregoing general description and the following detailed description are examples and explanatory and are not restrictive of the present disclosure, as claimed. It should be understood that the various embodiments are not limited to the arrangements and vascular access instrumentality shown in the drawings. Also, the drawings are not necessarily to scale. It should also be understood that the embodiments may be combined. For example, one or more features of a particular vascular access instrument may be combined with one or more features of another particular vascular access instrument. It should also be understood that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example vascular access system, illustrating an example vascular access instrument in an example retracted position, according to some embodiments;

FIG. 1B is an upper perspective view of the vascular access system, illustrating the vascular access instrument in an example advanced position, according to some embodiments;

FIG. 1C is an enlarged upper perspective view of a portion of the vascular access system, illustrating the vascular access instrument in the advanced position, according to some embodiments;

FIG. 1D is an upper perspective view of an example distal portion of the vascular instrument, according to some embodiments;

FIG. 2A is an upper perspective view of another vascular access system, illustrating the vascular access instrument in an example advanced position, according to some embodiments;

FIG. 2B is an upper perspective view of the distal portion of the vascular access instrument, illustrating an example U-shape, according to some embodiments;

FIG. 2C is an upper perspective view of the distal portion of the vascular access instrument, illustrating a bent portion that includes the U-shape and an example loop, according to some embodiments;

FIG. 3A is an upper perspective view of the distal portion of the vascular access instrument, illustrating an example distal end, according to some embodiments;

FIG. 3B is a side view of the distal portion of the vascular access instrument, illustrating the distal end of FIG. 3A, according to some embodiments;

FIG. 3C is another upper perspective view of the distal portion of the vascular access instrument, illustrating an example closed coil portion, according to some embodiments;

FIG. 3D is an upper perspective view of an example proximal portion of the vascular access instrument, illustrating an example straight portion, according to some embodiments;

FIG. 3E is an upper perspective view of the proximal portion of the vascular access instrument, illustrating the straight portion and an example core portion joined together, according to some embodiments;

FIG. 3F is an upper perspective view of the proximal portion of the vascular access instrument, illustrating the coil portion terminating at the core portion, according to some embodiments;

FIG. 3G is an upper perspective view of the proximal portion of the vascular access instrument, illustrating an example twist, according to some embodiments;

FIG. 3H is an upper perspective view of the proximal portion of the vascular access instrument, illustrating the straight portion and an example bend, according to some embodiments;

FIG. 3I is an upper perspective view of the coil portion, illustrating an example taper, according to some embodiments;

FIG. 3J is a side view of the coil portion, illustrating the taper, according to some embodiments;

FIG. 4A is an upper perspective view of the distal portion of the vascular access instrument, illustrating an absence of the core portion, according to some embodiments;

FIG. 4B is an upper perspective view of the proximal portion of the vascular instrument, illustrating the absence of the core portion, according to some embodiments;

FIG. 5 is an upper perspective view of an example tapered core portion, according to some embodiments;

FIG. 6 is an upper perspective view of the distal portion of the vascular access instrument, illustrating an example cap, according to some embodiments;

FIG. 7A is an upper perspective view of the proximal portion of the vascular access instrument, illustrating an example other wire, according to some embodiments;

FIG. 7B is an upper perspective view of the proximal portion of the vascular access instrument, illustrating an example tube, according to some embodiments; and

FIG. 7C is an upper perspective view of the proximal portion of the vascular access instrument, illustrating the tube joined to the straight portion and the core portion, according to some embodiments.

### DESCRIPTION OF EMBODIMENTS

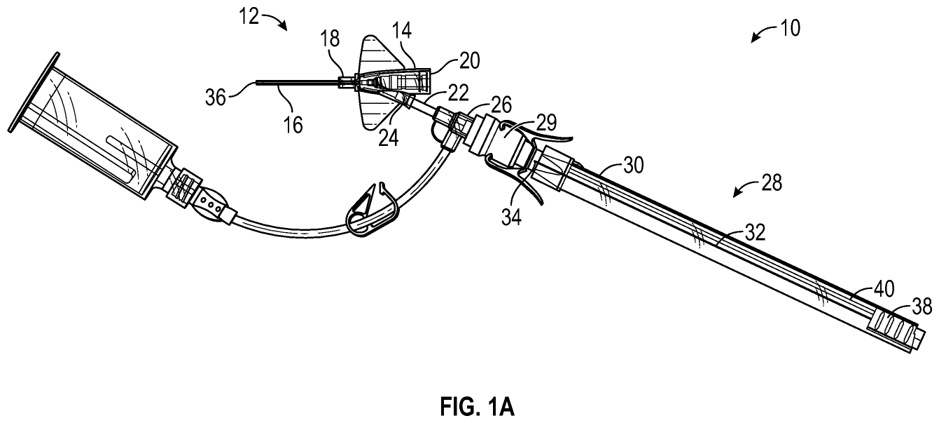

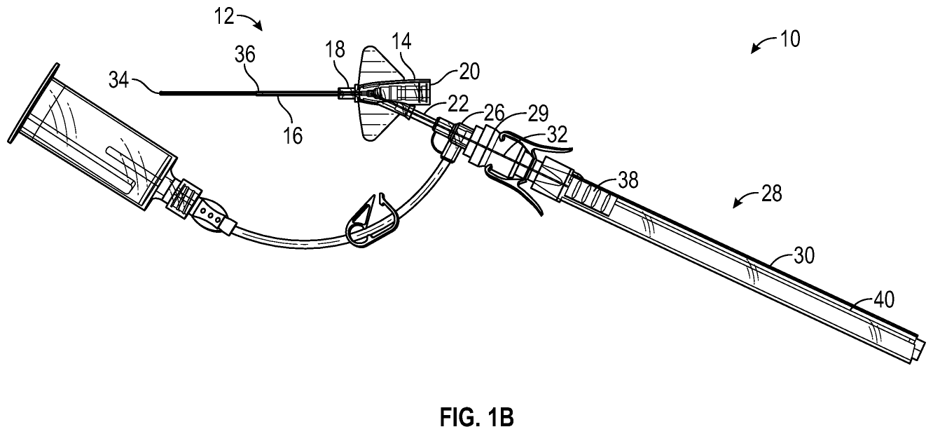

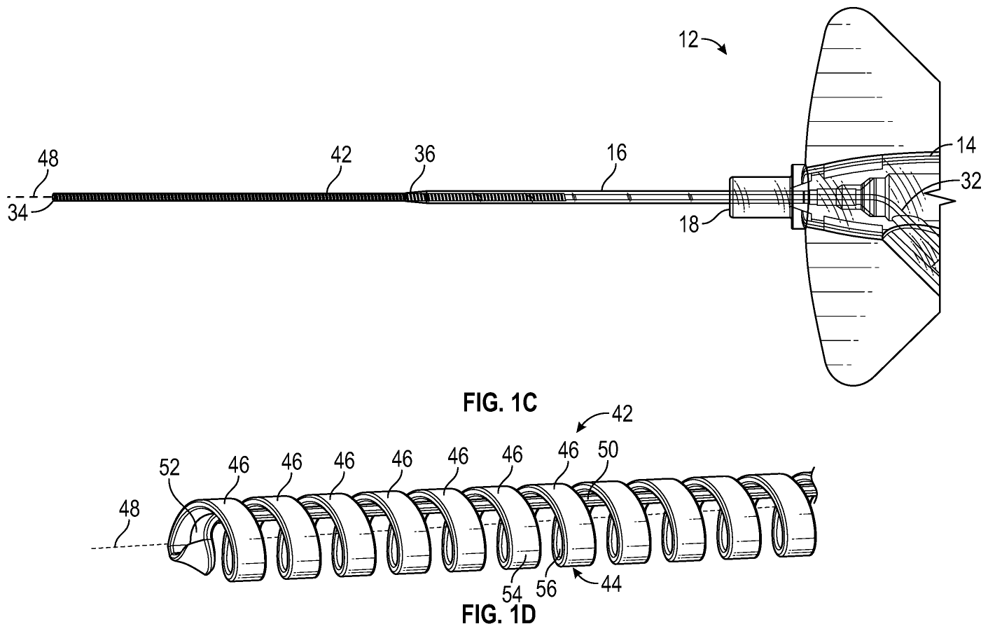

Referring now to FIGS. 1A-1B, a vascular access system 10 is illustrated, according to some embodiments. In some embodiments, the vascular access system 10 may include a catheter assembly 12, which may include a catheter adapter 14 and a catheter 16. In some embodiments, the catheter 16 may include a peripheral intravenous catheter, a peripherally-inserted central catheter, or a midline catheter. In some embodiments, the catheter adapter 14 may include a distal end 18, a proximal end 20, and a lumen extending through the distal end 18 and the proximal end 20. In some embodiments, the catheter 16 may extend distally from the distal end 18 of the catheter adapter 14.

In some embodiments, the catheter adapter 14 may be integrated with an extension tube 22, which may extend from a side port 24 of the catheter adapter 14. In some embodiments, an adapter 26, such as a Y-adapter or a T-adapter, for example, may be coupled to a proximal end of the extension tube 22.

In some embodiments, an instrument advancement device 28 may be coupled to the catheter assembly 12 in various ways. As an example, the instrument advancement device 28 may be coupled to a port of the adapter 26. As another example, the instrument advancement device 28 may be coupled to a needleless connector 29 disposed between the port of the adapter 26 and the instrument advancement device 28. As another example, the instrument advancement device 28 may be coupled to the proximal end 20 of the catheter adapter 14. In some embodiments, another extension tube and/or a blood collection device adapter may be coupled to another port of the adapter 26. In some embodiments, the blood collection device adapter may receive a blood collection device, such as, for example, a syringe or a blood collection tube.

In some embodiments, the instrument advancement device 28 may include a housing 30 configured to couple to the catheter assembly 12. In some embodiments, the instrument advancement device 28 may include a vascular access instrument 32. In some embodiments, the instrument advancement device 28 may include any suitable delivery device. Some examples of instrument advancement devices that may be used with the vascular access instrument 32 are described further in in U.S. patent application Ser. No. 16/037,246, filed Jul. 17, 2018, entitled “EXTENSION HOUSING A PROBE OR INTRAVENOUS CATHETER,” U.S. patent application Ser. No 16/388,650, filed Apr. 18, 2019, entitled “INSTRUMENT DELIVERY DEVICE HAVING A ROTARY ELEMENT,” U.S. patent application Ser. No. 16/037,319, filed Jul. 17, 2018, entitled “MULTI-DIAMETER CATHETER AND RELATED DEVICES AND METHODS,” U.S. patent application Ser. No. 16/502,541, filed Jul. 3, 2019, entitled “DELIVERY DEVICE FOR A VASCULAR ACCESS INSTRUMENT,” U.S. patent application Ser. No. 16/691,217, filed Nov. 21, 2019, entitled “SYRINGE-BASED DELIVERY DEVICE FOR A VASCULAR ACCESS INSTRUMENT,” U.S. patent application Ser. No. 16/742,013, filed Jan. 14, 2020, entitled “CATHETER DELIVERY DEVICE AND RELATED SYSTEMS AND METHODS,” and U.S. patent application Ser. No. 16/838,831, filed Apr. 2, 2020, entitled “VASCULAR ACCESS INSTRUMENT HAVING A FLUID PERMEABLE STRUCTURE AND RELATED DEVICES AND METHODS,” which are each incorporated by reference in their entirety.

In some embodiments, the instrument advancement device 28 may be configured to introduce the vascular access instrument 32 into the catheter assembly 12. In some embodiments, in response to the vascular access instrument 32 being introduced into the catheter assembly 12, the vascular access instrument 32 may access a fluid path of the catheter assembly 12 and/or the vascular access instrument 32 may extend through the catheter assembly 12 to access the vasculature of the patient.

In some embodiments, the instrument advancement device 28 may be configured to advance the vascular access instrument 32 between a retracted position, illustrated, for example, in FIG. 1A, to an advanced position, illustrated, for example, in FIG. 1B. In some embodiments, a distal end 34 of the vascular access instrument 32 may be disposed distal to a distal end 36 of the catheter 16 in response to the vascular access instrument 32 being in the advanced position. In some embodiments, in response to the vascular access instrument 32 being in the retracted position, the distal end 34 of the vascular access instrument 32 may be disposed within the housing 30. In some embodiments, a proximal end of the vascular access instrument 32 may be coupled to an advancement tab 38, which may be gripped and moved along a slot 40 by a user to move the vascular access instrument 32 between the retracted position and the advanced position. In some embodiments, the advancement tab 38 may extend through the slot 40, and a portion of the advancement tab 38 coupled to the proximal end of the vascular access instrument 32 may be within the housing 30.

In some embodiments, the catheter 16 may be constructed of fluorinated ethylene propylene, TEFLON™, silicon, thermoplastic elastomer, thermoplastic polyurethane, a fluorinated polymer, a hydrophilic material, a hydrophobic material, an anti-fouling material, or another suitable material. In some embodiments, the catheter 16 may include an anti-thrombogenic coating. In some embodiments, all or a portion of the vascular access instrument 32 may be constructed of metal or another suitable material.

Referring now to FIGS. 1C-1D, in some embodiments, the vascular access instrument 32 may include a wire 42, which may be monolithically formed as a single unit. In some embodiments, the vascular access instrument 32 may include no more than one wire 42, which may be continuous. Prior art vascular access instruments may be constructed of four or more components, which may be coupled together. In some embodiments, the wire 42 that is monolithically formed as a single unit may facilitate increased fluid flow rates through the vascular access instrument 32 and into the catheter 16 and less shear stress on fluid moving through the vascular access instrument 32.

In some embodiments, the wire 42 may include a coil portion 44, which may include multiple loops 46 wound around a central axis 48. In some embodiments, the wire 42 may include a core portion 50, which may extend through the coil portion 44. In some embodiments, the core portion 50 may be straight and aligned with the central axis 48. In some embodiments, the wire 42 may include a bent portion 52 connecting a distal end of the coil portion 44 with a distal end of the core portion 50.

In some embodiments, the bent portion 52 may form the distal end 34 of the wire 42. In some embodiments, the bent portion 52 may be disposed distal to the coil portion 44. In some embodiments, the distal end 34, which may include a distal-most surface of the wire 42, may be blunt. In some embodiments, the distal end 34 may facilitate soft and gentle contact with a wall of the vasculature in response to insertion of the vascular access instrument 32 into the vasculature. In some embodiments, the distal end 34 and/or the coil portion 44 may reduce shear stress on fluid moving through the vascular access instrument 32.

In some embodiments, the coil portion 44 may be formed by a flat portion of the wire 42 wound around the central axis 48 into the loops 46. As referred to in the present disclosure, the term “flat portion of the wire” may correspond to a portion of the wire 42 that includes a first side 54 and a second side 56 opposite the first side 54, and the first side 54 and/or the second side 56 is planar prior to the wire 42 being wound around the central axis 48 into the loops 46 during manufacture. In some embodiments, the first side 54 may form an outer surface of the coil portion 44. In some embodiments, the second side 56 may form an inner surface of the coil portion 44.

In some embodiments, the flat portion may increase an inner diameter of the coil portion 44 to facilitate an increase fluid flow rate through the vascular access instrument 32. In some embodiments, the flat portion may increase the inner diameter of the coil portion 44 and still allow an outer diameter of the coil portion 44 to be approximately equal to an outer diameter of a standard vascular access instrument. In some embodiments, dimensions of the coil portion 44 may vary based on a gauge-size of the catheter 16, a stiffness of the vascular access instrument 32, a spacing between each of the loops 46 of the coil portion 44, a number or size of fluid pathways along the length of the vascular access instrument 32, or another factor. In some embodiments, a pitch of the coil portion 44 may vary along a length of the coil portion 44.

In some embodiments, the core portion 50 may contact the inner surface of the coil portion or be spaced apart from the coil portion. In some embodiments, the core portion 50 may be coupled to the inner surface of the coil portion 44. However, in some embodiments, due to the wire 42 being monolithically formed as a single unit, the core portion 50 may not be coupled to the inner surface of the coil portion 44 but may still be secure and provide structural support to the vascular access instrument 32.

In some embodiments, each of the loops 46 of the coil portion 44 may be spaced apart from a next adjacent loop of the loops 46, which may facilitate fluid permeability of the vascular access instrument 32. In these embodiments, the coil portion 44 may be referred to as open. In some embodiments, the coil portion 44 may provide multiple and/or continuous fluid pathways along a length of the vascular access instrument 32, which may facilitate entry of blood into the catheter assembly 12 from a portion of the vasculature more distant from the catheter 16. In some embodiments, the coil portion 44 and the fluid pathways along the length of the vascular access instrument 32 may facilitate increased flow rates of fluid through the vascular access instrument 32 and the catheter 16. In some embodiments, the coil portion 44 and the fluid pathways along the length of the vascular access instrument 32 may facilitate a reduced blood collection time. In some embodiments, the coil portion 44 may reduce a shear stress and related risk of hemolysis of blood moving into and/or through the catheter 16.

In some embodiments, the coil portion 44 and/or the core portion 50 may be cylindrical. In some embodiments, the core portion 50 may be flat, which may increase flow through the coil portion 44 and/or the catheter 16. In some embodiments, the core portion 50 may be offset from the central axis 48, which may facilitate blood flow into the catheter assembly 12 and/or fluid infusion into the vasculature along the central axis 48 and a central portion of the coil portion 44 and/or a central portion of the catheter 16.

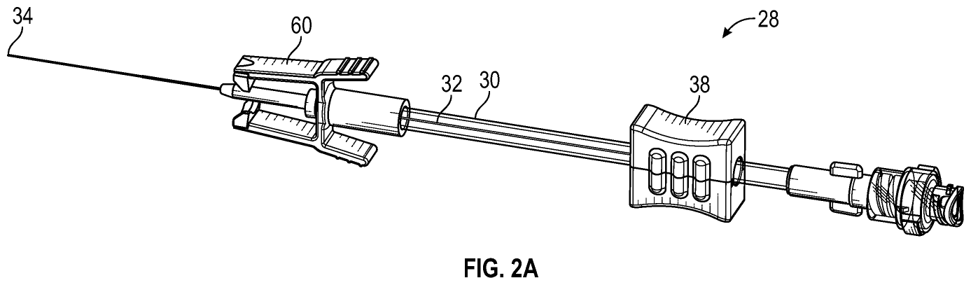

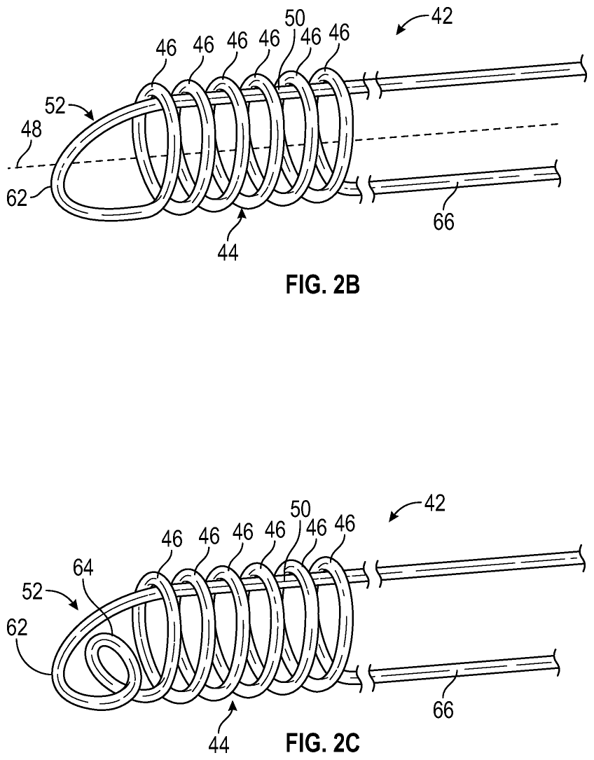

Referring now to FIG. 2A, the instrument advancement device 28 may include the housing 30, which may include an extension tube. In some embodiments, the instrument advancement device 28 may be configured to couple to the catheter assembly 12 via a distal connector 60, which may include a luer adapter and/or one or more lever arms. In some embodiments, the instrument advancement device 28 may include the vascular access instrument 32. In some embodiments, a proximal end of the vascular access instrument 32 may be coupled to a housing or an advancement tab 38, which may be gripped and moved along the housing 30 to move the vascular access instrument 32 between the retracted position and the advanced position. In some embodiments, the housing 30 may facilitate movement of the vascular access instrument 32 without direct contact by the user, such as via pinching of the proximal end of the vascular access instrument 32, translation of one or more ball bearings along the housing 30, or another suitable mechanism.

Referring now to FIG. 2B, in some embodiments, the bent portion 52 may include a U-shape 62, which may be blunt and atraumatic. In some embodiments, the U-shape 62 may be generally aligned with the central axis 48. Referring now to FIG. 2C, additionally or alternatively, in some embodiments, the bent portion 52 may include one or more loops 64, which may be blunt and atraumatic. In some embodiments, the wire 42 may include a straight portion 66 connected to a proximal end of the coil portion 44. In some embodiments, the straight portion 66 may be parallel to the core portion 50. In some embodiments, the straight portion 66 and the core portion 50 may be coupled to a particular advancement tab of a particular instrument advancement device.

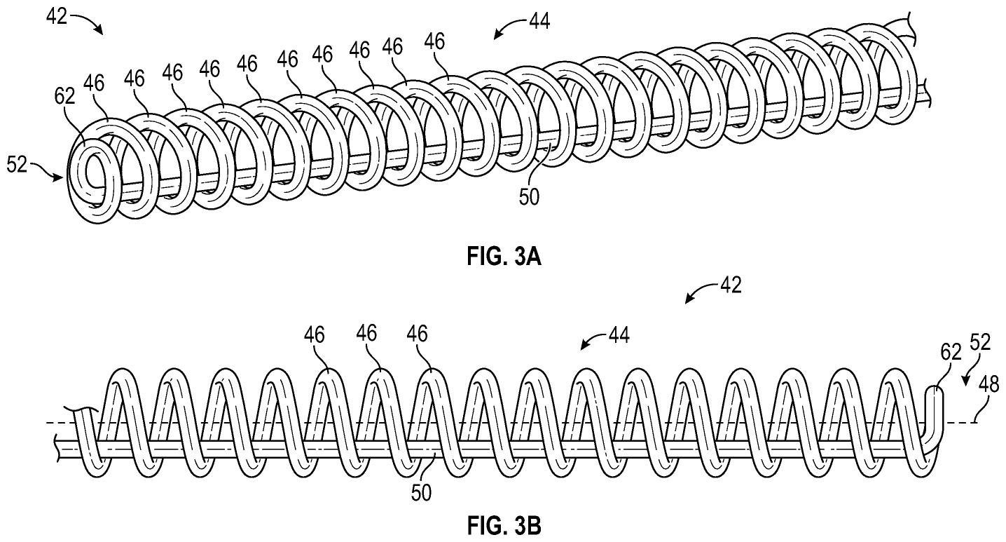

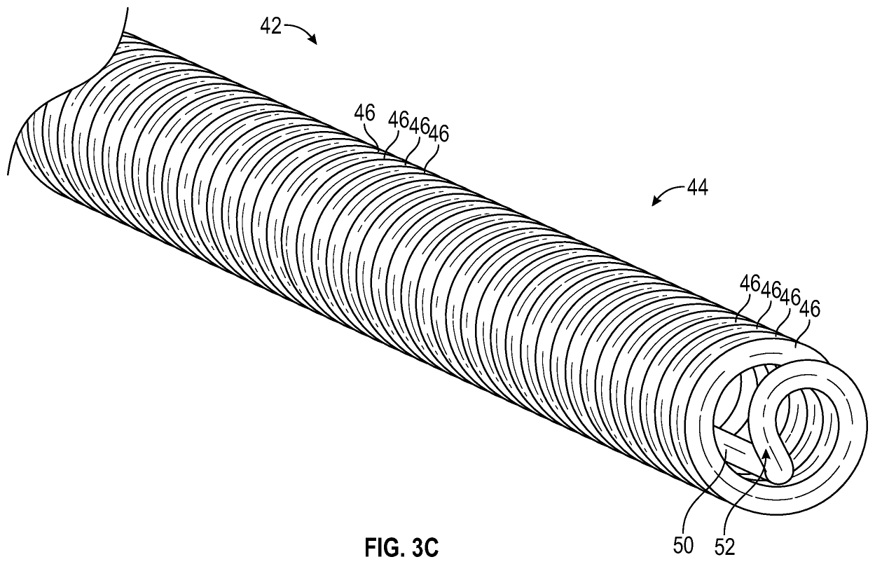

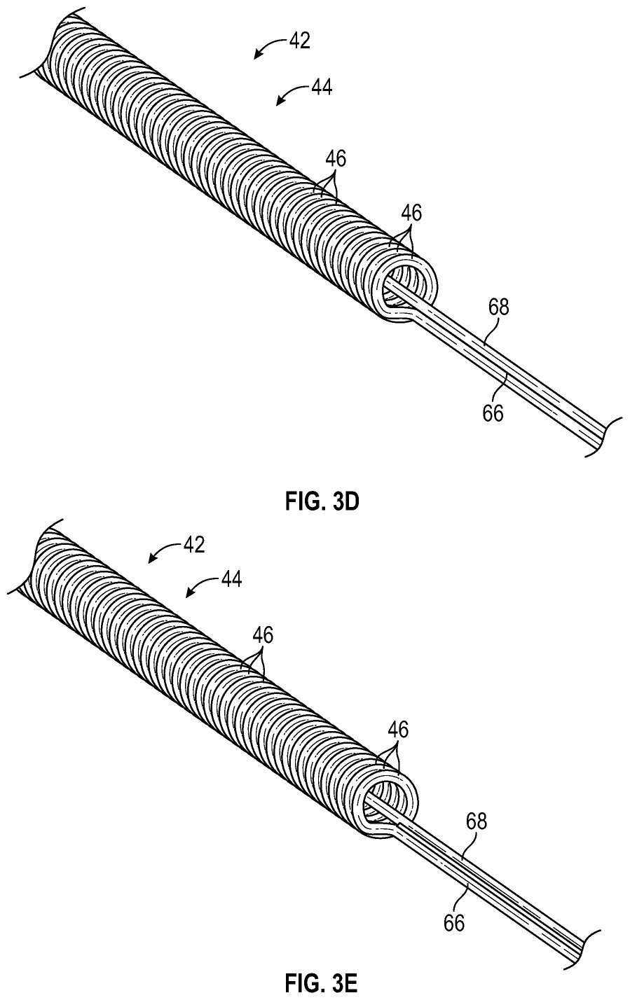

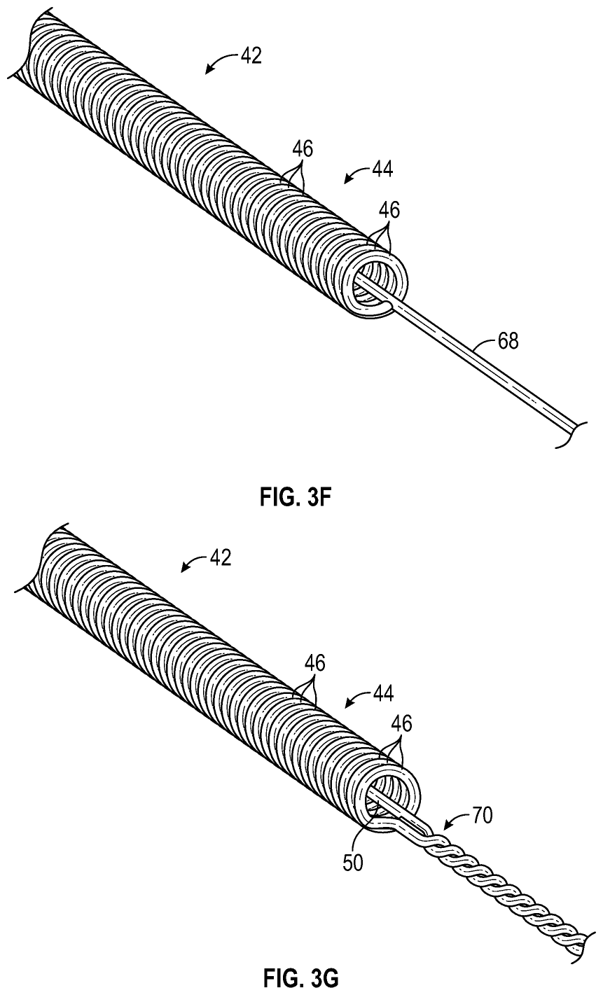

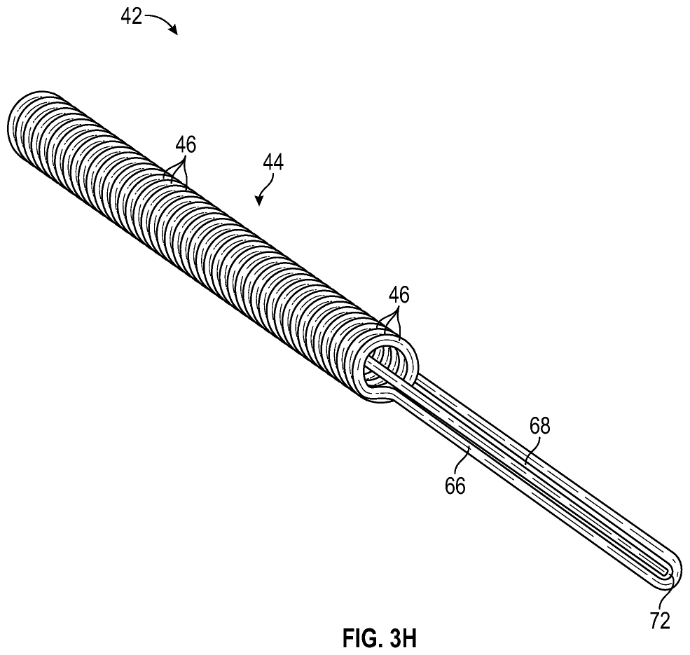

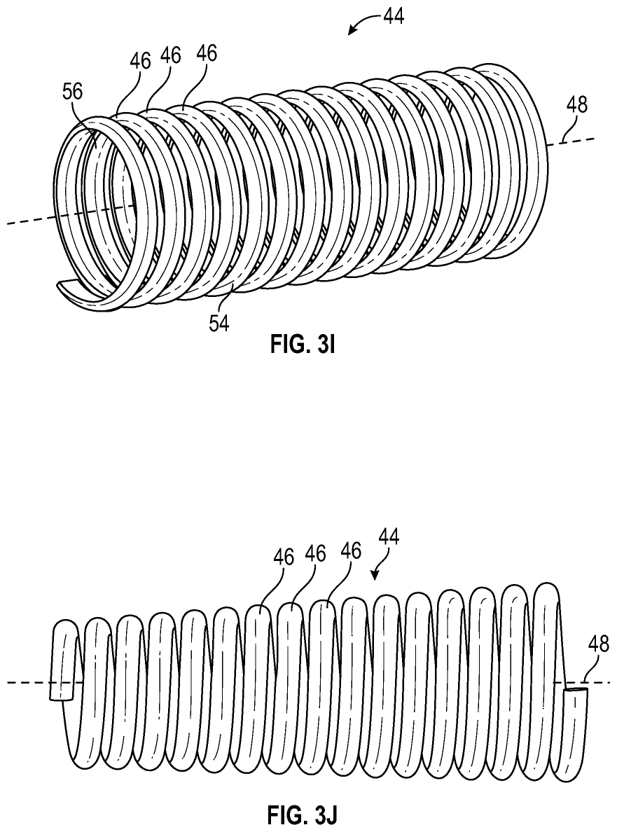

Referring now to FIGS. 3A-3B, in some embodiments, the U-shape 62 may be generally perpendicular to the central axis 48, which may facilitate a decreased risk of injury to the vasculature. Referring now to FIG. 3C, in some embodiments, each of the loops 46 of the coil portion 44 may contact the next adjacent loop of the loops 46 around a circumference of the next adjacent loop. Thus, in some embodiments, the coil portion 44 may be referred to as closed, as little or no fluid may pass between adjacent coils.

Referring now to FIG. 3D, in some embodiments, the straight portion 66 may be parallel to a proximal end 68 of the core portion 50. In some embodiments, the inner portion of the straight portion 66 and the inner portion of the proximal end 68 of the core portion 50 may not be joined together. In some embodiments, the proximal end 68 of the core portion 50 may be disposed proximal to the coil portion 44.

Referring now to FIG. 3E, in some embodiments, an inner portion of the straight portion 66 and an inner portion of the proximal end 68 of the core portion 50 may be joined together for axial stiffness. For example, the inner portion of the straight portion 66 and the inner portion of the proximal end 68 of the core portion 50 may be joined together via an adhesive, welding, coating, or any other suitable joining methods.

Referring now to FIG. 3F, in some embodiments, a proximal end of the coil portion 44 may be joined to the core portion 50.

Referring now to FIG. 3G, in some embodiments, the vascular access instrument 32 may include a twist 70 proximal to the coil portion 44 and/or the core portion 50. In some embodiments, one end of the twist 70 may extend from the coil portion 44 and another end of the twist 70 may extend from the core portion 50.

Referring now to FIG. 3H, in some embodiments, at a proximal end of the straight portion 66 there may be a bend 72 and/or the vascular access instrument 32 may include one or more additional bends, which may form loops around the proximal end 68 of the core portion 50 for added strength and stiffness. In some embodiments, the bend 72 and/or the additional bends may be about 180 degrees. In some embodiments, the bend 72 may provide three parallel wire portions for added stiffness. In some embodiments, each of the additional bends may provide an additional parallel wire section for additional stiffness.

Referring now to FIGS. 3I-3J, in some embodiments, the coil portion 44 may be tapered. As illustrated, for example, in FIGS. 3I-3J, the coil portion 44 may be tapered outwardly in the proximal direction.

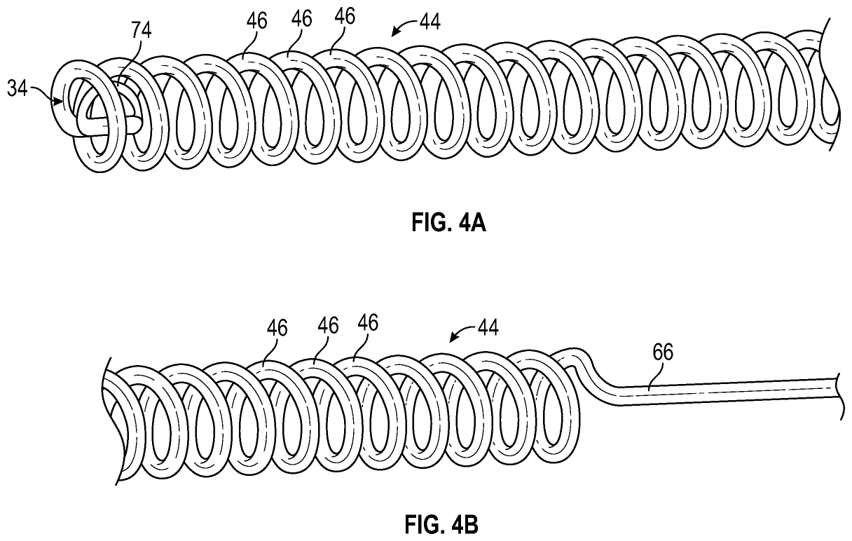

Referring now to FIGS. 4A-4B, in some embodiments, the vascular access instrument 32 may not include the core portion 50, increasing flow through the coil portion 44 and/or the catheter 16. In these embodiments, the distal end 34 may include one or more other loops 74, which may be oriented in a different direction from the loops 46 of the coil portion 44 and may ensure an atraumatic tip. In some embodiments, there may be a ball or cap disposed at the distal end 18.

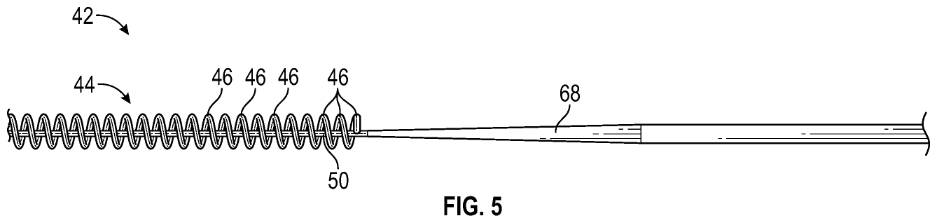

Referring now to FIG. 5, in some embodiments, the proximal end 68 of the core portion 50 may taper outwardly in a proximal direction, which may provide increased stiffness to the vascular access instrument 32.

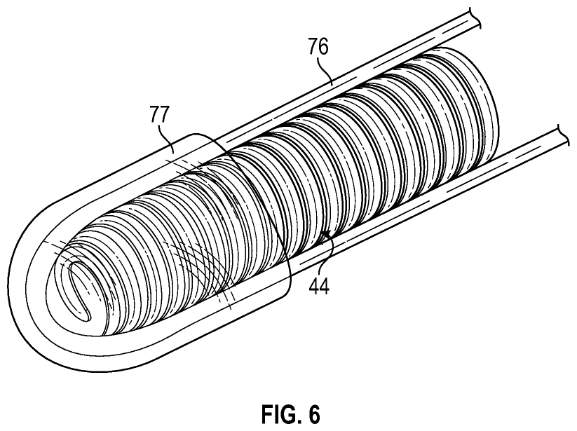

Referring now to FIG. 6, in some embodiments, a U-shaped wire 76 may extend around the coil portion 44 and may be disposed distal to the coil portion 44. In some embodiments, a cap 77 may be disposed around a distal end of the U-shaped wire 76 and a distal end of the coil portion 44, which may facilitate a decreased risk of injury to the vasculature. In some embodiments, the cap 77 may be joined to the U-shaped wire 76 via an adhesive, welding, coating, or any other suitable joining methods. In some embodiments, ends of the U-shaped wire 76 and/or the coil portion 44 may be coupled to a particular advancement tab of a particular instrument advancement device.

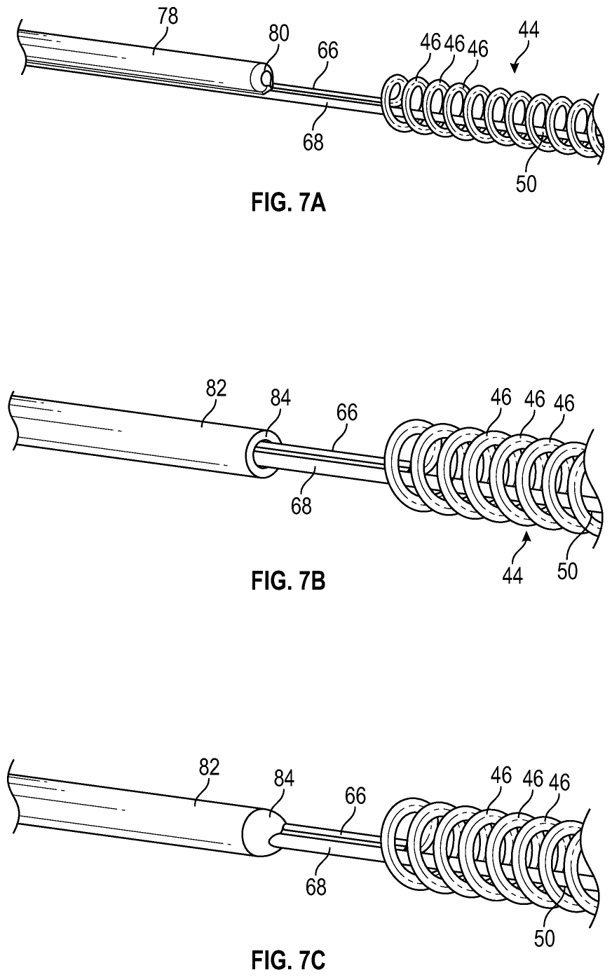

Referring now to FIG. 7A, in some embodiments, the vascular access instrument 32 may include another wire 78 coupled to the straight portion 66 and the proximal end 68 of the core portion 50. In some embodiments, the other wire 78 may provide added support to one or more of the proximal end 44, the straight portion 66, and the proximal end 68 of the core portion 50. In some embodiments, the other wire 78 may include nitinol, stainless steel, or another suitable material. In some embodiments, one or more of the coil portion 44, the straight portion 66, the bent portion 52 (see, for example, FIGS. 1D and 2B-3C) and the core portion 50 may be constructed of nitinol, stainless steel, carbon steel, steel, chrome, or another suitable material. In some embodiments, one or more of the coil portion 44, the straight portion 66, the bent portion 52, and the core portion 50 may include a coating, such as, for example, TEFLON™ or parylene. In some embodiments, a distal end 80 of the other wire 78 may be disposed proximal to the coil portion 44.

Referring now to FIGS. 7B-7C, in some embodiments, the vascular access instrument 32 may include a tube 82 surrounding the straight portion 66 and/or the proximal end 68 of the core portion 50. In some embodiments, the tube 82 may provide added support to the wire 42. In some embodiments, the tube 82 may include nitinol, stainless steel, polymeric tubing, polyimide, coating, shrink tubing, or another suitable material. In some embodiments, a distal end 84 of the tube 82 may be disposed proximal to the coil portion 44. As illustrated in FIG. 7C, in some embodiments, the tube 82 may be joined to the straight portion 66 and/or the proximal end 68 of the core portion 50 such as by an adhesive, welding, coating, or any other suitable joining method.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

We claim:

1. A vascular access instrument configured to insert through a vascular access device, the vascular access instrument comprising:
a wire that is monolithically formed as a single unit, the wire comprising:
a coil portion, comprising a plurality of loops wound around a central axis;
a core portion extending through the coil portion and aligned with the central axis; and
a bent portion connecting a distal end of the coil portion with a distal end of the core portion, wherein the bent portion forms a distal end of the wire.

2. The vascular access instrument of claim 1, wherein the bent portion is disposed distal to the coil portion.

3. The vascular access instrument of claim 2, wherein the bent portion comprises a U-shape.

4. The vascular access instrument of claim 3, wherein the bent portion comprises a loop.

5. The vascular access instrument of claim 1, wherein the coil portion is formed by a flat portion of the wire wound around the central axis into the plurality of loops.

6. The vascular access instrument of claim 1, wherein each of the plurality of loops of the coil portion is spaced apart from a next adjacent loop of the plurality of loops. The vascular access instrument of claim 1, wherein each of the plurality of loops of the coil portion contacts a next adjacent loop of the plurality of loops around a circumference of the next adjacent loop.

8. The vascular access instrument of claim 1, wherein the coil portion and the core portion are cylindrical.

9. The vascular access instrument of claim 2, wherein the core portion is offset from the central axis.

10. The vascular access instrument of claim 1, wherein the flat portion of the wire comprises a first side and a second side opposite the first side, wherein the first side forms an outer surface of the coil portion, wherein the second side forms an inner surface of the coil portion, wherein the core portion contacts the inner surface of the coil portion.

11. The vascular access instrument of claim 1, wherein the distal end of the wire is blunt.

12. The vascular access instrument of claim 1, wherein the wire further comprises a straight portion connected to a proximal end of the coil portion, wherein the straight portion is parallel to a proximal end of the core portion.

13. The vascular access instrument of claim 12, wherein an inner portion of the straight portion and an inner portion of the proximal end of the core portion are joined together.

14. The vascular access instrument of claim 12, wherein an inner portion of the straight portion and an inner portion of the proximal end of the core portion are not joined together.

15. The vascular access instrument of claim 12, further comprising another wire coupled to the straight portion and the proximal end of the core portion, wherein the other wire comprises nitinol or stainless-steel, wherein a distal end of the other wire is disposed proximal to the coil portion.

16. The vascular access instrument of claim 12, further comprising a tube surrounding the straight portion and the proximal end of the core portion, wherein the tube comprises nitinol or stainless-steel, wherein a distal end of the tube is disposed proximal to the coil portion.

17. The vascular access instrument of claim 16, wherein the tube is joined to the straight portion and the proximal end of the core portion.

18. The vascular access instrument of claim 1, wherein a proximal end of the coil portion is joined to the core portion.

19. The vascular access instrument of claim 1, wherein a proximal end of the core portion tapers outwardly in a proximal direction.

20. A vascular access system, comprising:
a catheter assembly, comprising a catheter adapter and a catheter extending distally from the catheter adapter;
an instrument advancement device coupled to the catheter assembly, wherein the instrument advancement device comprises a vascular access instrument, wherein the instrument advancement device is configured to advance the vascular access instrument from a retracted position to an advanced position beyond a distal end of the catheter, wherein the vascular access instrument comprises:
a wire that is monolithically formed as a single unit, the wire comprising:
a coil portion, comprising a plurality of loops wound around a central axis;
a core portion extending through the coil portion and aligned with the central axis; and
a bent portion connecting a distal end of the coil portion with a distal end of the core portion, wherein the bent portion forms a distal end of the wire.
